(19) United States
(12) Patent Application Publication
(10) Pub. No.: US 2009/0069560 A1
(43) Pub. Date: Mar. 12, 2009

(54) Process For Obtaining Levofloxacin Free From Salts

(75) Inventors: Francisco Palomo Nicolau, Alcala de Henares (ES); Antonio Cosme Gomez, Torres De Alameda (ES); Rafael Martin Sanchez, Villalbilla (ES)

Correspondence Address: WOLF, BLOCK, SHORR AND SOLIS-COHEN LLP, 250 PARK AVENUE, 10TH FLOOR, NEW YORK, NY, 10177, US

(73) Assignee: Quimica Sintetica, S.A., Barcelona (ES)

(21) Appl. No.: 11/813,146

(22) PCT Filed: Dec. 15, 2005
(86) PCT No.: PCT/IB2005/003924

§ 371 Date: Jun. 16, 2008

(30) Foreign Application Priority Data
Dec. 31, 2004 (ES) P-200403166

(51) Int. Cl. C07D 498/06

(52) U.S. Cl. 544/101

(57) ABSTRACT

This invention relates to a process for obtaining levofloxacin free from salts. In this process the starting product used is the compound (V), alkaline hydrolysis of which within a water-(C₁-C₄)alcohol mixture, and subsequent neutralisation and separation of the salts, leads to levofloxacin free from salts, without need to carry out any final step of extraction and without using halogenated solvents.

One characteristic of the process described is that no extractions are necessary in the final step of the process.

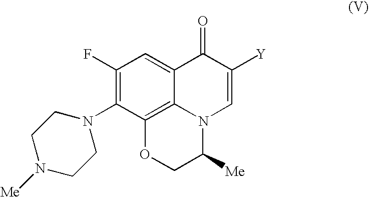

### FIELD OF THE INVENTION

The present invention relates to a process for preparing levofloxacin free from salts.

### STATE OF THE PRIOR ART

Levofloxacin is the international nonproprietary name of (S)-9-fluoro-3-methyl-10-(4-methyl-1-piperazinyl)-7-oxo-2,3-dihydro-7H-[1,2,3-de]benzoxazine-6-carboxylic acid of formula (I), with application in medicine and known for its antimicrobial activity:

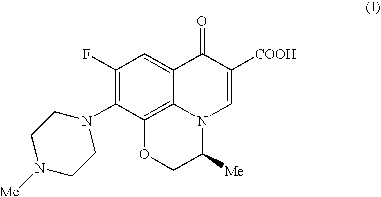

Levofloxacin crystallises in hemihydrated form or as a monohydrate, which last differs from the former in the number of molecules of water in the crystal, and in the form of anhydrous crystals produced by dehydration of these hemihydrates and monohydrates.

European patent EP 444 678 B1 discloses a process for preparing hemihydrate and monohydrate levofloxacin that substantially consists of controlling the water content of a solution of a water-miscible aqueous solvent that contains levofloxacin, such that in order to obtain the hemihydrate the water content has to be 10% or less by volume, while to obtain the monohydrate it has to have more than 10% water. Among the miscible aqueous solvents the patent mentions ethanol, methanol, propanol and acetone.

European patent application EP 1 367 132A1 discloses a process of preparing levofloxacin that consists of a total of ten steps, the last of which includes a hydrolysis of the compound (II), wherein R⁶ is a C₁-C₆ alkyl, a BZ₂ group wherein Z represents a halogen atom, a C₁-C₆ alkoxy group or a C₂-C₇ alkylcarbonyloxy group, as shown below:

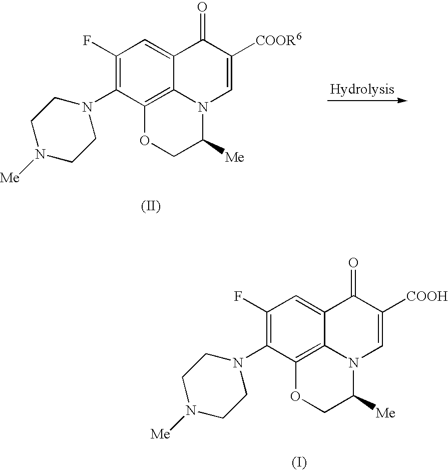

This hydrolysis step can be carried out, for example, by heating the compound (II) in the presence of a base in a protonic solvent, e.g. in the presence of trialkylamine in an alcoholic solvent. More specifically, the hydrolysis reaction can be carried out merely by heating the compound (II) in the presence of triethylamine in ethanol. This process involves a laborious purification process through to obtaining the final product. Thus, following the alkaline hydrolysis step the reaction mixture is evaporated to dryness, the residue is dissolved in dilute HCl, CHCl₃ is added and the phases are separated. The aqueous layer is adjusted to pH 11 with NaOH and then to pH 7.4 with HCl. The resulting mixture is extracted with CHCl₃. The extracts are combined and dried over sodium sulphate and the chloroform is distilled. The resulting solid is recrystallised in ethanol-ether.

In an academic paper, Hiroshi et al. in Chem. Pharm. Bull. 1986 34 (10) 4098-4102 describe a process of preparing ofloxacin (IV) whose last step comprises cyclation of the oxazine ring and subsequent alkaline hydrolysis. According to the authors, the process described is applicable to any of the optically active isomers of ofloxacin simply by using (R) or (S) alaninol as one of the starting products of the process. Levofloxacin is the levo-form isomer of ofloxacin.

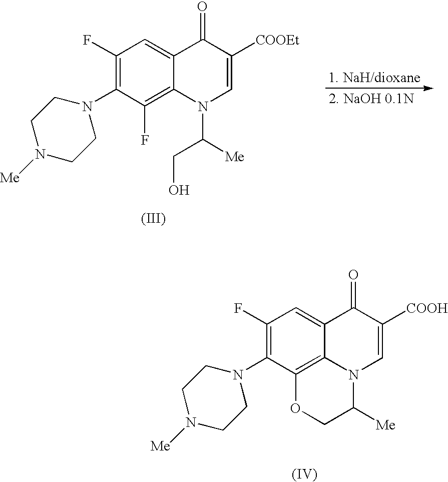

In this process the ofloxacin is isolated after a process of neutralisation with acetic acid, extraction with chloroform and recrystallisation of a chloroform-ethanol mixture.

Applied at an industrial level for manufacturing levofloxacin, these processes of alkaline hydrolysis and subsequent neutralisation involve a number of extractions in the final step. In these extractions, the organic phase has an interface that gives rise to the presence of salts and water in the product obtained. The presence of salts originates problems of crystallisation and low titrations in the solid obtained, thus rendering it necessary to reprocess the product obtained. The use of halogenated solvents also constitutes a problem from the ecological point of view, due to their highly polluting effect.

These disadvantages—complex purification process, low yields, use of halogenated solvents and eventual reprocessing—hinder and render more expensive the production of levofloxacin at industrial level.

There therefore exists a need to have a process for preparing levofloxacin free from salts with good chemical yield, without the need to carry out large numbers of extractions and substantially avoiding the use of halogenated solvents that pollute the environment.

The authors of the present invention have discovered a process for obtaining levofloxacin free from salts that does not require extractions to be carried out in the final step of purification.

### OBJECT OF THE INVENTION

The object of the present invention is a process for obtaining levofloxacin free from salts that does not require extractions be carried out in the final step of purification.

### DETAILED DESCRIPTION OF THE INVENTION

The process object of the invention includes the hydrolysis of a compound (V)

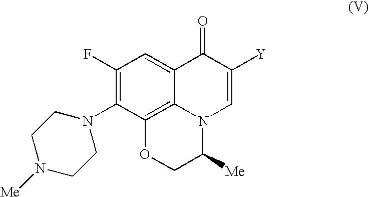

in which Y is either a COOR₁ group in which R₁ is a C₁-C₆ alkyl, or a BZ₂ group in which Z represents a halogen atom, a C₁-C₆ alkoxy group or a C₂-C₇ alkylcarbonyloxy group, or Y is a nitryl group within a water-(C₁-C₄) alkylic alcohol mixture, subsequent neutralisation, separation of the salts, followed by isolation of levofloxacin.

The process is characterised in that all the steps are carried out in the water-(C₁-C₄)alcohol mixture and in that after the neutralising agent has been added said water-(C₁-C₄)alcohol mixture must comprise proportions between 13.5 and 16% of water expressed in (w/w) or 12.2 and 14.7% of water expressed in (v/v).

The compound (V) can be prepared, for example, according to the process described in the academic publication by Hiroshi et al.

The hydrolysis reaction can be carried out by heating the product (V) in the presence of a base, such as sodium hydroxide or potassium hydroxide or any other organic or inorganic base. The C₁-C₄ alkyl alcohol can be selected from the group of compounds such as methanol, ethanol, isopropanol, 1-propanol, 1-butanol, isobutanol, sec-butanol and tert-butanol. Preferably, the C₁-C₄ alcohol is ethanol.

The neutralising agents can be selected from proton acids such as hydrochloric acid, acetic acid, sulphuric acid, phosphoric acid and nitric acid, or an ionic-exchange resin such as Dowex HCR-S Resin can be used, too. Preferably, the neutralising agent used is sulphuric acid.

The C₁-C₄ alkyl alcohol can be selected from the group of compounds such as methanol, ethanol, isopropanol, 1-propanol, 1-butanol, sec-butanol and tert-butanol. Preferably, the C₁-C₄ alcohol is ethanol.

The compound (V) reacts with the alkaline agent at a temperature between 70 and 80° C., within the water-(C₁-C₄)alcohol mixture for at least 1 hour.

The reaction is carried out in 10-15 volumes, preferably in 11-12 volumes of an alcohol/water mixture in relation to the product (V), using between 1 and 3, preferably between 1 and 2 equivalents, and more preferably 1.10 equivalents of the base.

Once the reaction has finished, the product is treated with active carbon, is filtered and a neutralising agent is added. The neutralising agent is added in a proportion of equivalent to equivalent in relation to the added base. The salts are separated, for example, by filtration at a temperature between 70 and 75° C., and the product (I) precipitates by cooling down to 0-5° C.

After adding the neutralising agent, the water-(C₁-C₄)alcohol mixture must comprise proportions of 13.5 to 16% of water expressed in (w/w) or 12.2 and 14.7% of water expressed in (v/v).

The precipitate obtained is levofloxacin (I), which is filtered, washed with the cold C₁-C₄ alcohol and dried at 40° C. to constant weight.

The crude levofloxacin so obtained can later be recrystallised in an ethanol-water mixture. For example, the water content in the mixture can range between 13.5 and 16% expressed in (w/w) or 12.2 and 14.7% expressed in (v/v), preferably between 14.8 and 16% expressed in (w/w) or 13.5 and 14.7% expressed in (v/v), such that levofloxacin hemihydrate is obtained.

Surprisingly, it has been found that the new process permits levofloxacin to be obtained with a yield exceeding 70% on the basis of compound (V), without need to carry out any final step of extraction and without using halogenated solvents, with a purity and quality suitable for the preparation of pharmaceutical formulations and with a salt content not exceeding 0.1%.

The content of salts in levofloxacin is determined by the “Residue on Ignition” process of Farmacopea Americana USP 27, equivalent to the “Sulphated Ash” process of the Farmacopea Europea, 4th edition.

The examples that follow are outlined solely for the purposes of providing a skill in the art with a detailed explanation of the process object of the invention.

### EXAMPLE 1

50 g (0.1278 moles, 1 equivalent) of compound V (Y=COOEt) are placed in a flask, stirred into a mixture of 500 ml of ethyl alcohol, 82 ml of water and 5.5 g (0.137 moles) of sodium hydroxide. This is heated at reflux for 1 hour, cooled, treated with active carbon and filtered, washing the cake with 50 ml of ethyl alcohol. The temperature is adjusted to 50-60° C. and 6.7 g (0.068 moles) of pure sulphuric acid are added. The mixture is heated to 70-75° C. and the insoluble salts formed are removed by filtration, washing the cake with 50 ml of ethyl alcohol. The water content of the mixture must then range between 13.5 and 16% w/w (12.2 and 14.7% v/v). The filtrate obtained is cooled down to 0-5° C. and the precipitate obtained is washed with ethyl alcohol and dried, to give 35-36 g of pure levofloxacin hemihydrate. The process yield is 75%. The content in salts is lower than 0.10%.

### EXAMPLE 2

50 g of compound V (Y=COOEt) (0.1278 moles) are placed in a flask and stirred into a mixture of 500 ml of ethyl alcohol, 82 ml of water and 5.5 g (0.137 moles) of sodium hydroxide. This is heated at reflux for 1 hour, cooled, treated with active carbon and filtered, washing the cake with 50 ml of ethyl alcohol. 8.2 g (0.137 moles) of pure acetic acid are added. The water content of the mixture must then range between 13.5 and 16% w/w (12.2 and 14.7% v/v). The precipitate obtained is cooled down to 0-5° C., filtered, washed with ethyl alcohol and dried, to give 30-31 g of pure levofloxacin hemihydrate. The process yield is 64%. The content in salts is lower than 0.10%.

### EXAMPLE 3

50 g of compound V (Y=COOEt) (0.1278 moles) is placed in a flask and stirred into a mixture of 500 ml of ethyl alcohol, 82 ml of water and 5.5 g (0.137 moles) of sodium hydroxide. This is heated at reflux for 1 hour, cooled, treated with active carbon and filtered, washing the cake with 50 ml of ethyl alcohol. 12.2 g (0.137 moles) of 65% nitric acid are added. The water content of the mixture must then range between 13.5 and 16% w/w (12.2 and 14.7% v/v). The precipitate obtained is cooled down to 0-5° C., filtered, washed with ethyl alcohol and dried, to give 22-23 g of pure levofloxacin hemihydrate. The process yield is 47%. The content in salts is lower than 0.10%.

### EXAMPLE 4

50 g of compound V (Y=COOEt) (0.1278 moles) is placed in a flask, stirred into a mixture of 500 ml of ethyl alcohol, 50 ml of water and 5.7 g (0.14 moles) of sodium hydroxide. This is heated at reflux for 1 hour, cooled, treated with active carbon and filtered, washing the cake with 50 ml of ethyl alcohol. The temperature is adjusted to 50-60° C. and 35 g of Dowex HCR-S Resin are added. The mixture is heated to 70-75° C. and the Dowex Resin is filtered, washing the cake with 50 ml of ethyl alcohol. The water content of the mixture must then be adjusted to between 13.5 and 16% w/w (12.2 and 14.7% v/v) using the necessary amount of water. The mixture so obtained is cooled down to 0-5° C. and the precipitate obtained is washed with ethyl alcohol and dried, to give 33.5 g of pure levofloxacin hemihydrate. The process yield is 70%. The content in salts is lower than 0.10%.

1. Process for obtaining levofloxacin containing no more than 0.1% in salts, that includes the hydrolysis of compound (V), in the presence of a base, 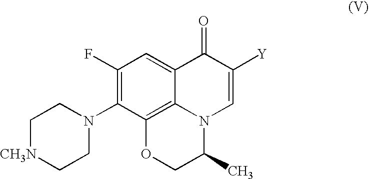
in which Y is either a COOR₁ group in which R₁ is a C₁-C₆ alkyl, or a BZ₂ group in which Z represents a halogen atom, a C₁-C₆ alkoxy group or a C₂-C₇ alkylcarbonyloxy group, or Y is a nitrile group within a water-(C₁-C₄)alcohol mixture, with subsequent neutralisation, separation of the salts, followed by the isolation of levofloxacin, without need to carry out any final step of extraction and without using halogenated solvents.

2. Process according to claim 1, wherein all the steps are carried out in the water-(C₁-C₄)alcohol mixture and in that after the neutralising agent has been added said water-(C₁-C₄)alcohol mixture must comprise proportions between 13.5 and 16% of water expressed in (w/w) or 12.2 and 14.7% of water expressed in (v/v).

3. Process according to claim 1, wherein the levofloxacin is isolated by precipitation of the mixture of water and a C₁-C₄ alcohol by cooling to between 0-5° C.

4. Process according to claim 1, wherein the hydrolysis reaction is carried out when the product (V) is heated in the presence of an organic or inorganic base, preferably an inorganic base, more preferably sodium hydroxide or potassium hydroxide, at a temperature between 70 and 80° C.

5. Process according to claim 1, wherein the C₁-C₄ alcohol can be selected from the group methanol, ethanol, isopropanol, 1-propanol, 1-butanol, sec-butanol and tert-butanol, preferably ethanol.

6. Process according to claim 1, wherein the reaction is carried out in 10-15 volumes of a water-(C₁-C₄)alcohol mixture in relation to the product (V), preferably in 11-12 volumes, using between 1 and 3 equivalents of the base, preferably between 1 and 2 and more preferably still 1.10 equivalents in relation to the product (V).

7. Process according to claim 1, wherein the neutralising agent can be selected from proton acids such as hydrochloric acid, acetic acid, sulphuric acid, phosphoric acid and nitric acid, or an ionic-exchange resin such as Dowex HCR-S Resin, preferably the neutralising agent used is sulphuric acid and the neutralising agent is added in a proportion of equivalent to equivalent in relation to the added base.

8. Process according to claim 1, wherein in the compound (V) Y is equal to COOC₂H₅, the base is sodium hydroxide or potassium hydroxide and is used in a proportion of 1.10 equivalents in relation to equivalent of compound (V), the C₁-C₄ alcohol is ethanol, the water-ethanol mixture comprises proportions between 13.5 and 16% of water expressed in (w/w) or 12.2 and 14.7% of water expressed in (v/v), between 11 and 12 volumes of the water/ethanol mixture in relation to product (V) are used, the neutralising agent is sulphuric acid in a proportion of equivalent to equivalent in relation to the added base, the salts are separated by filtration at a temperature between 70 and 75° C. and the hemihydrate levofloxacin product precipitates by cooling down to 0-5° C.
